(19) United States
(12) Patent Application Publication
(10) Pub. No.: US 2005/0092328 A1
(43) Pub. Date: May 5, 2005

(54) Endotracheal tube holder with an adjacent feeding tube holder for neo-natal use

(76) Inventors: Brieanna Herrick, Sandy, UT (US); Kent Herrick, Sandy, UT (US); Brent Herrick, Sandy, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/697,711

(22) Filed: Oct. 30, 2003

(51) Int. Cl. A62B 9/06; A61M 16/00

(52) U.S. Cl. 128/207.17

(57) ABSTRACT

A method and apparatus for providing an endotracheal tube holder which prevents injury to a neo-natal patient, including a flexible arcuate face plate, an tube holding member that is placed in the face plate in front of the patient's mouth, and an attachment mechanism which does not cause elastic compression on the neo-natal patient. A bite block is provided for preventing damage caused by the neo-natal patient biting on the endotracheal tube, the tube holding member is able to adjust to different sizes of endotracheal tubes to thereby hold them firm, an additional tube can be added for simultaneous access to the patient's mouth, cheek pads prevent injury to the patient's cheeks, and integral eye protection is provided on the attachment device.

### BACKGROUND OF THE INVENTION

1. Field of the Invention

This invention relates generally to medical equipment. More specifically, the invention relates to an endotracheal tube holder which is specially adapted for use in neo-natal care.

2. Description of Related Art

An endotracheal tube holder is used during various medical procedures. An endotracheal tube is inserted through a patient's mouth, and into the trachea. The purpose of such intubations may be to ensure proper ventilation, or for other reasons. Other tubes may also be inserted, such as a feeding tube. When these tubes are to remain in the patient for a period of time, it is common to anchor the tubes to the patient in some manner. For example, medical tape can be applied directly to the patient's face. There also appear to be an assortment of head and neck braces which hold a mouthpiece against the patient's mouth, as will now be discussed.

The state of the art in endotracheal tube holders is replete with different designs intended to address different issues. For example, some of the designs are intended, at least partially, to address the issue of attachment to a patient. Others are designed for more securely holding the endotracheal tube so that it does not slip out of the trachea. Others are intended to reduce the damage caused to patients by extended attachment of tubes via medical tape on the patient's face. It is useful to examine what some of these prior references teach in order to illustrate their shortcomings.

There are at least several endotracheal tube holders that are relevant to this disclosure. For example, U.S. Pat. No. 5,551,421 issued to Noureldin et al. teaches an endotracheal tube holder with a ratcheting device for positioning the tube away from the center of the patient's mouth to prevent obstruction in the flow of regurgitated food or fluid. The tube holder includes straps for securing it to the patient's neck.

U.S. Pat. No. 5,555,881 issued to Rogers et al. describes an Endotracheal tube holder that has a collar member for securely holding the tube, and a brace coupled to the collar member that holds the tube in front of the patient's mouth. The brace is disposed between the patient's upper lip and nose, and the collar member extends downwards therefrom. Alternative embodiments address others means for holding the tube. Teeth within the collar member can grip and hold a tube inserted therethrough.

U.S. Pat. No. 5,490,504 issued to Vrona et al. teaches an endotracheal tube holder and a holding device for securing the holder to the patient with a neck brace. The lateral position of the endotracheal tube is modifiable so that it can be moved to one side for access.

U.S. Pat. No. 5,419,319 issued to Werner teaches an endotracheal tube holder comprising a rigid face plate which conforms to the patient's mouth. The tube holder hangs from the face plate and in front of the patient's mouth. The position of the tube holder is adjustable in front of the patient's mouth, and holes for straps are provided in the mouthpiece to attach it to the patient's head.

U.S. Pat. No. 5,368,024 issued to Jones teaches a system for holding an endotracheal tube in front of a patient's mouth. The system comprises disks which are stuck to the patient's cheeks on either side of the mouth, and straps are provided for providing pull and counter-pull on the disks to hold them in place. The tube holder comprises an open ring that can be pulled apart to insert the tube, and which will pinch the tube when released.

U.S. Pat. No. 5,345,931 issued to Battaglia, Jr. teaches an endotracheal tube holder with provides a rigid and conforming face piece which has a tube holder that can be adapted to different sizes of tubes. Straps are secured to the rigid face plate to hold it in place to the patient's neck. The tube holder fits in a track of the rigid face plate, and can slide within the track to hold the tube in any position horizontally in front of the patient's mouth.

U.S. Pat. No. 5,402,776 issued to Islava teaches an endotracheal tube holder which provides a means for securing the tube in front of the patient's mouth by a clamp coupled to a rigid face plate. The face plate is secured by a strap around the patient's head.

There are several important disadvantages to the endotracheal tube holders described in the patents above. First, all of these endotracheal tube holders are dangerous for neo-natal use. There are special considerations when using an endotracheal tube holder for small infants. For example, their skin is very sensitive. Medical tape can cause a rash, or even a more severe allergic reaction. The skin can even be damaged directly when the medical tape is removed. It is noted that the Jones patent is the only one which shows an endotracheal tube holder attached to a young person. The remaining endotracheal tube holders either show the device coupled to an adult, or do not show the device attached to a person. Furthermore, the Jones patent teaches an adhesive disk that is secured to the patient's cheek.

Another reason that the prior art patents are dangerous for neo-natal use is that the neck and head braces would probably do damage to the neck and/or head of a neo-natal infant. This is because of the extremely delicate nature of a neo-natal patient's neck and head structure. Any type of applied pressure is undesirable because it can interfere with the normal expansion and reshaping of cranial bones after birth. The prior art references fail to teach the need or the ability to provide an attachment system which can provide secure attachment of an endotracheal tube holder to a neo-natal patient without risking damage to the patient. Furthermore, the references also fail to teach an attachment system which can be quickly and easily adjusted for size and fit, and even replaced.

Another danger that is not addressed by the prior art references is the need for eye protection. Neo-natal patients in particular are very susceptible to retinal damage because of the harsh visible or ultraviolet lighting conditions that are often necessary for warmth, visual monitoring, or medical treatment. The prior art references fail to demonstrate the need or ability to provide eye protection.

The prior art references fail to teach the need for or the ability to provide an endotracheal tube holder which can also accommodate another tube. For example, feeding tubes are sometimes used at the same time as an endotracheal tube. The feeding tube is also put into the patient's mouth.

Another danger that is not addressed by the prior art references is the importance of providing some means for preventing damage a patient's mouth. The gums and teeth of a neo-natal patient are particularly susceptible to damage from constant contact with an object that is forcing it to conform to the shape of some other object. In particular, the endotracheal tube or the endotracheal tube holder can cause damage. The prior art references fail to demonstrate the need for or the ability to provide some protection for the gums and teeth.

The prior art references fail to demonstrate the need for or the ability to provide some protection for the patient's cheeks. The references typically show a face plate assembly that rests directly on the patients cheeks, or even worse, is attached to the cheeks by adhesive.

Therefore, it would be an advantage over the prior art to provide an endotracheal tube holder which could be secured to the neo-natal patient without having to use a brace that would apply pressure to the infant's head and/or neck. Essentially, the attachment system does not use any elastic compression to secure the endotracheal tube holder to the patient. It would be a further advantage to provide an endotracheal tube holder that can be quickly and easily adjusted in size, replaced, provide adjustable eye protection, enable simultaneous placement of more than one tube into the patient's mouth, and provide protection to the patient's gum, teeth, cheeks, and mouth.

### BRIEF SUMMARY OF THE INVENTION

It is an object of the present invention to provide a method and apparatus for holding an endotracheal tube stationary relative to a mouth of a neo-natal patient.

It is another object to provide an endotracheal tube holder which can be held stationary relative to the mouth without having to use an elastic attachment mechanism.

It is another object to provide an endotracheal tube holder which can be held stationary relative to the mouth without having to use an adhesive attachment mechanism.

It is another object to provide an attachment mechanism for the endotracheal tube holder to which adjustable eye protection can be attached.

It is another object to provide an endotracheal tube holder which enables simultaneous anchoring of a second tube relative to the patient's mouth.

It is another object to provide an endotracheal tube holder which also provides protection to a neo-natal patient's gums and teeth.

It is another object to provide an endotracheal tube holder which provides protection to a neo-natal patient's mouth structure.

It is another object to provide an endotracheal tube holder and attachment mechanism that will not interfere with normal expansion and reshaping of cranial bones after birth.

It is another object to provide an endotracheal tube holder with an attachment mechanism that is quickly and easily adjustable or removable.

The presently preferred embodiment of the present invention is an endotracheal tube holder comprising a flexible arcuate face plate, a tube holding member disposed in the face plate in front of the patient's mouth, and a means for attachment which does not cause elastic compression on the neo-natal patient. A bite block is provided for preventing damage caused by the neo-natal patient biting on the endotracheal tube, the tube holding member is able to adjust to different sizes of endotracheal tubes to thereby hold them firm, an additional tube can be added for simultaneous access to the patient's mouth, cheek pads prevent injury to the patient's cheeks, and integral eye protection is provided on the means for attachment.

The face plate curves around the patient's face in front of the mouth. On the ends of the face plate are a plurality of attachment slots in the two ends of the arcuate face plate to which attachment straps are coupled and extend around the patient's head to thereby secure the arcuate face plate to the patient, and a ratcheting endotracheal tube holder which is disposed within a U-shaped receptacle at a midpoint of the arcuate face plate.

In a first aspect of the invention, the ratcheting endotracheal tube holder which clamps around the endotracheal tube is comprised of a cylindrical length of rigid material. The cylindrical material is then caused to pivot open. The endotracheal tube is disposed within a depression within the cylindrical material, and the cylindrical material is then caused to pivot shut around the endotracheal tube.

In a second aspect of the invention, a ratcheting mechanism causes the cylindrical material to close around the endotracheal tube at varying degrees of tightness. The cylinder is then disposed within the U-shaped receptacle at the midpoint of the face plate.

In a third aspect of the invention, a second hole is provided in the endotracheal tube holder through which another tube can be disposed.

In a fourth aspect of the invention, a non-elastic headband is provided with a hook and loop fastening system such as straps having VELCRO(™) to give it sufficient structure such that the face plate can be held in place in front of the patient's mouth. The hook and loop fastening system of VELCRO(™) enables the head band to be quickly and easily adjusted for proper fit, or removal from the face plate.

In a fifth aspect of the invention, a tubular member extends from the U-shaped receptacle and into the patient's mouth as a bite block to prevent damage to the patient's teeth and gums as they bite on the endotracheal tube holder.

In a sixth aspect of the invention, cheek pads are disposed under the attachment ends of the face plate to prevent them from injuring the patient's cheek and cheek bones.

In a seventh aspect of the invention, an adjustable eye covering is coupled to the attachment straps to thereby protect the patient's eyes from surrounding light sources.

These and other objects, features, advantages and alternative aspects of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is a perspective elevational view of the components of the presently preferred embodiment which is constructed in accordance with the principles of the present invention.

FIG. 2A is a front elevational view of the face plate of the presently preferred embodiment, which illustrates the U-shaped receptacle and the attaching arms.

FIG. 2B is a top elevational view of the face plate shown in FIG. 2A.

FIG. 2C is a side elevational profile view of the face plate shown in FIG. 2A.

FIG. 3 is a side elevational and cut-away profile view of the presently preferred embodiment of the tube holding member by which the endotracheal tube is held.

FIG. 4A is an elevational profile view of the tube holding member of FIG. 3 taken along the perspective of A-A.

FIG. 4B is an elevational profile view of the tube holding member of FIG. 4A with the top member pivoted away from the bottom member.

FIG. 5 is an elevational top view of an alternative embodiment of the tube holding member with a bite block extending inward towards the patient's mouth.

FIG. 6 is an elevational profile view of a patient with the endotracheal tube holding system attached to the patient using a preferred arrangement of attaching straps.

FIG. 7 is an elevational profile view of a patient with the endotracheal tube holding system attached to the patient in an alternative arrangement of attaching straps, and with an optional eye protection system for shielding the eyes of neo-natal patients.

FIG. 8A is a top elevational view of a cheek pad that surrounds the attaching arm of the face plate to protect the patient's cheeks.

FIG. 8B shows the cheek pad of FIG. 8A and its relative position with respect to the attaching arm before the hook and loop fastening system of VELCRO(™) straps are folded over

### DETAILED DESCRIPTION OF THE INVENTION

Reference will now be made to the drawings in which the various elements of the present invention will be given numerical designations and in which the invention will be discussed so as to enable one skilled in the art to make and use the invention. It is to be understood that the following description is only exemplary of the principles of the present invention, and should not be viewed as narrowing the claims which follow.

It is useful to have an overview of the present invention before delving into the detailed description of the preferred embodiment. Accordingly, it is observed that the present invention advantageously provides features of an endotracheal tube holder which are uniquely adapted to the special needs of a neo-natal patient. However, these special adaptations are also applicable to young children or adult patients. Therefore, the principles described hereinafter find application to patients of any age group. Nevertheless, the advantages of the present invention will be described from the perspective of the special needs of the neo-natal patient.

The advantageous features to be described are designed with the purpose of safeguarding very sensitive bone and tissue of a neo-natal patient. However, even though neo-natal patients are particularly vulnerable, everyone undergoing medical care appreciates careful handling. It is unfortunate that patients sometimes suffer injuries as a result of the care they are receiving. The injuries are typically considered to be unavoidable.

The presently preferred embodiment of the present invention is designed to accomplish the same purposes as the medical equipment it is designed to replace, while at the same time, and provide a new level of comfort and protection against the injuries that the medical equipment can cause.

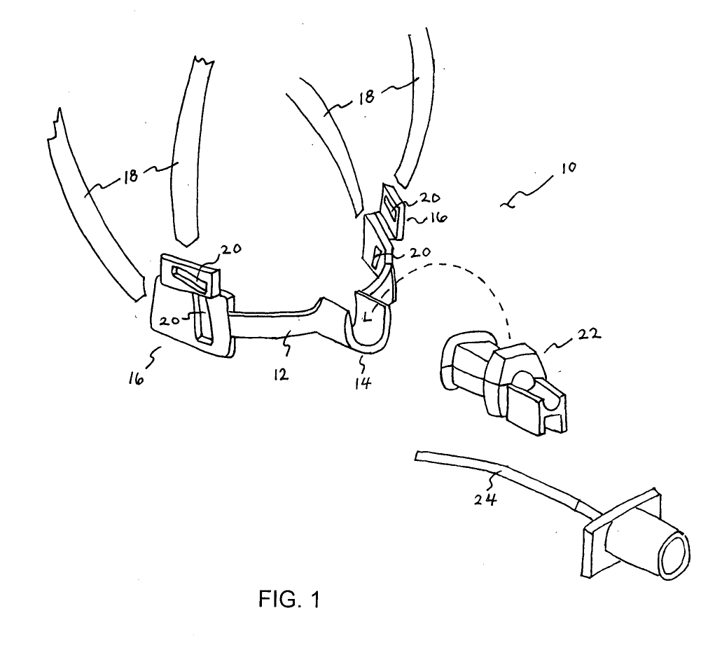

FIG. 1 is provided as an overview of the general operation of the presently preferred embodiment of the present invention. This figure shows that the invention is comprised of three separate components which are combined to create an attachable endotracheal tube holding system. In this view, the components are shown in perspective to better illustrate the relationships between them.

The first component is the arcuate face plate 12 of the endotracheal tube holder 10 which forms the framework of the endotracheal tube holding system. The face plate 12 is comprised of a semi-flexible arcuate material which conforms to the curvature of a patient's face at the mouth, and extends backwards along the patient's cheeks. The face plate 12 has a U-shaped receptacle 14 centered in a midpoint of the face plate 12. The open end of the U-shaped receptacle 14 is open upwards relative to the face plate 12.

The face plate 12 also has two attaching ends 16. The attaching ends 16 are each designed to receive attachment straps 18, the ends of which are partially shown in FIG. 1. In this presently preferred embodiment, the attachment straps 18 are coupled to slots 20 in the attaching ends 16 by The hook and loop fastening system of VELCRO(™). The U-shaped receptacle 14 is made of a generally rigid material, but is sufficiently flexible so as to allow a tube holding member 22 to slide therein and snap securely into place.

The tube holding member 22 is designed to open along its length, allowing an endotracheal tube 24 to be disposed therein, and to then be closed tightly around the endotracheal tube 24, leaving sufficient space for air or other fluids to pass therethrough. The tube holding member 22 is then disposed within the U-shaped receptacle 14 and held securely.

The endotracheal tube 24 is inserted into the patient's trachea, and the endotracheal tube holder 10 is positioned on the patient's face in front of the mouth. The attachment straps 18 are then coupled to the face plate 12 and adjusted so as to pull the face plate 12 gently but firmly against the patient's mouth so that the endotracheal tube 24 cannot be dislodged from the patient's trachea.

More detailed figures of the components described generally in FIG. 1 will enable the user to better understand the specific benefits of the endotracheal tube holder 10 described above.

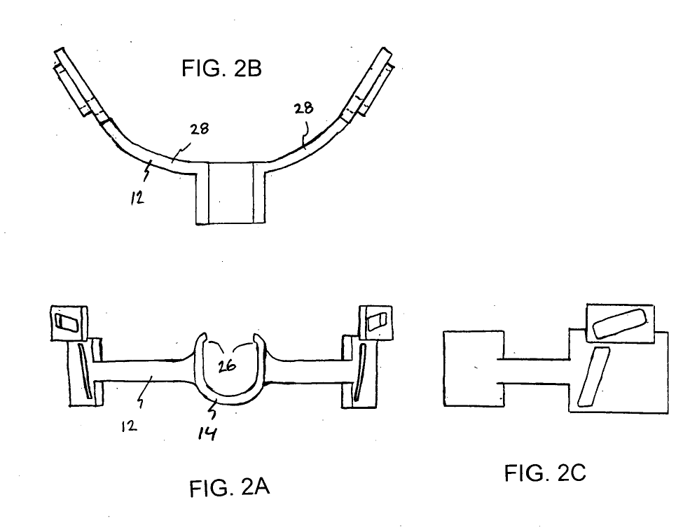

FIG. 2A is a front profile elevational view of the face plate 12. It is apparent that the U-shaped receptacle 14 has two upper lips 26 which help to retain the tube holding member 22 (FIG. 1) after it is inserted.

FIG. 2B is a top profile bird's eye view of the face plate 12 looking down from above. The flexible arms 28 are able to bend and conform to the patient's cheeks.

FIG. 2C is a side profile elevational view of face plate 12. The slots 20 are clearly visible. It should be remembered that the slanting of the slots is provided to assist in attaching the attachment straps 18 to the face plate 12. However, these slots 18 can be slanted in any preferable direction, in accordance with the requirements of the particular attachment straps 18 being used.

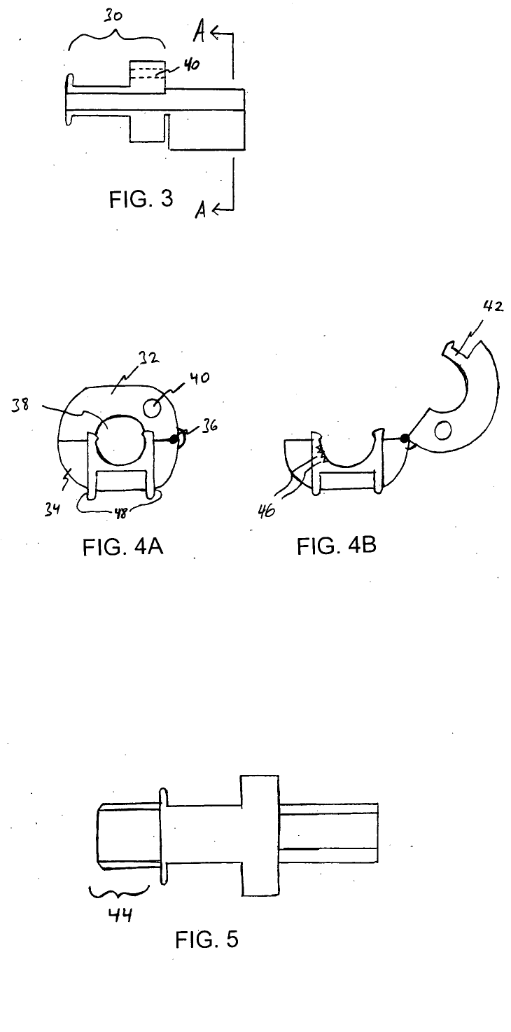

FIG. 3 is a cut-away elevational profile view of the tube holding member 22 which holds the endotracheal tube 24 (FIG. 1). In the presently preferred embodiment, the tube holding member 22 is shown as only wrapping around the endotracheal tube along a portion 30 of its length. Specifically, the portion 30 which wraps around the endotracheal tube 24 is disposed within the U-shaped receptacle 14 of the face plate 12.

FIG. 4A is an end elevational profile view of the tube holding member 22 as seen from the perspective A-A shown in FIG. 3. The tube holding member 22 has a top half 32, a bottom half 34, a hinge 36, and the hole 38 for the endotracheal tube 24. In an alternative embodiment, a second hole 40 is shown as a bore through the tube holding member 22. The hole 40 is large enough to fit a second tube for passing fluids, such as through a feeding tube. It is observed that inserting a feeding tube through the hole 40 and into the patient's mouth had advantages over a tube inserted through the nasal passages. For example, nasal passage damage is avoided, and the patient is able to breathe without an obstruction in the nasal passage.

The hole 40 can also be used to insert a cleaning tube. A cleaning tube is inserted to remove secretions from the patient's throat.

The tabs 46 are provided for releasing the ratchet hook 42 (FIG. 4B). Gently pressing on the tabs 46 causes the ratchet hook 42 to be released from the complementary receiving indentations 46 (FIG. 4B).

FIG. 4B is an illustration of the tube holding member 22 shown in FIG. 4A, but in an open position. What is important to recognize in this illustration is the locking mechanism. It is advantageous to provide a locking mechanism which is adjustable to the particular diameter of the endotracheal tube that is being held. Accordingly, it is preferable to utilize some type of ratchet mechanism as shown. The top half 32 has a ratcheting hook 42 which can be pressed into various and deeper complementary receiving indentations 46 in the bottom half 34. In this way, the endotracheal tube is always held tightly, regardless of its diameter.

It should be recognized that the ratcheting hook and receiving indentations are only an example of how the tube holding members 22 is able to grasp the endotracheal tube. This particular method has the advantage of being easily adjustable, but there are other methods which can also be used.

Holding the endotracheal tube is an important feature. If the endotracheal tube is free to move within the patient's mouth, the endotracheal tube can rub against the palate causing serious injury. Likewise, it is important that the face plate be held securely. Otherwise, the movement of the endotracheal tube holder can cause damage to the teeth and gums.

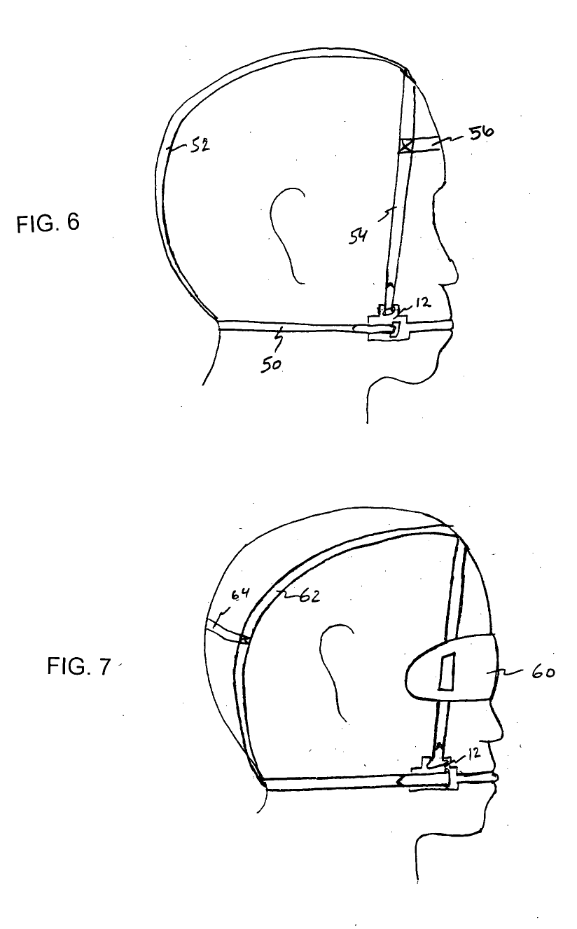

An alternative embodiment of the tube holding member 22 is shown in FIG. 5. FIG. 5 is a top elevational view which shows that an end of the tube holding member now extends into the mouth of the patient. This extension or bite block 44 is then covered by a sleeve of soft rubber. The bite block functions as a pacifier for the neo-natal infant. It also protects the gums from the harder material of the endotracheal tube that is inserted through the tube holding member 22. It is noted that prolonged pressure on the palate of the upper mouth can cause cleft palate. The soft rubber sleeve spreads out the area of contact between the teeth, gums and the endotracheal tube. This important because infants will use the endotracheal tube as a pacifier, causing a condition known as palatal groove which can extend to the alveolar ridge. This can affect the development of the lateral incisors, causing the condition Hypoplasia.

Another advantage of the present invention is that is addresses the need for visual monitoring of sores that can develop on the lips and the corners of the mouth. The face plate is a clear polycarbonate material, enabling health care workers to easily inspect the patient, and treat the conditions as soon as they are recognizable.

Finally, the details of the attachment straps 18 illustrated in FIGS. 6 and 7 should only be considered to be some examples of the possible configurations that they can form. In this preferred embodiment, however, they provide a distinct advantage over the prior art. Specifically, the attachment straps 18 are constructed of a soft and non-elastic material. The material is formed from strips so that the material is padded inside for added comfort as the patient lays on them. Preferably, the strips are sewn together at stress joints, thus only requiring The hook and loop fastening system of VELCRO(™) in order to tighten the whole attachment structure. Constructing the strips from a non-elastic material is important for reducing unwanted stresses on the cranial bones of the neo-natal patient.

FIG. 6 illustrates the presently preferred embodiment of the attachment straps 18. This configuration shows that a first attachment strap 50 extends around the base of the neck. A pair of straps 52 (only one shown) then form a V by traveling up from the base of the neck and over the top of the head until they meet at a strap 54 that extends upwards from the face plate 12. A stabilizing strap 56 extends across the forehead and is coupled at both ends to strap 54.

FIG. 7 is provided to show an alternative embodiment. An important new element is the eye protection 60 that is disposed on the eyes. This eye protection 60 also serves the function of stabilizing the straps on the head. The change that is also shown in the straps 62 that extend from the base of the neck to the forehead, and across the back of the head with strap 64 are only illustrative of the numerous modifications in shape of the straps that are within the scope of this invention. It is critical that the straps be non-elastic, and yet padded for comfort. The hook and loop fastening system of VELCRO(™) on the ends of the straps where they are coupled to the face plate enables the straps to be adjusted according to the dimensions of the head around which they are placed.

Another alternative embodiment of the present invention is a modification to the face plate 12. No matter how flexible or rigid the face plate 12 is constructed, a neo-natal patient will probably be laying at least partially on the attaching ends. The attaching ends are necessarily constructed of a durable plastic material. Accordingly, the attaching ends are likely to “dig into” the patient's cheeks and cheek bones. This is not only painful, but it can deform the cranial bones. In the alternative embodiment, the attaching ends are padded. This padding can be added in many ways.

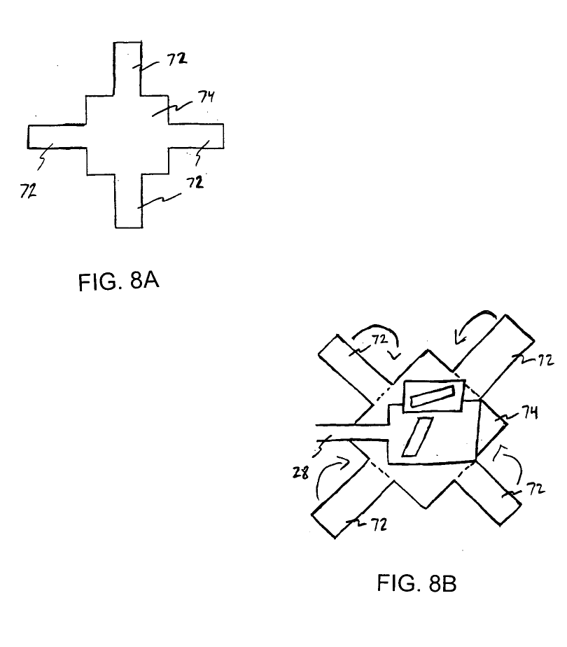

FIG. 8 is provided as an illustration of a cheek pad 70. A cheek pad 70 that is constructed using The hook and loop fastening system of VELCRO(™) can be used to wrap around the ends of the flexible arms 28. A simple embodiment of the cheek pad 70 is shown having four The hook and loop fastening system of VELCRO(™) arms 72 and a padded area 74. The cheek pad 70 slips underneath the attaching end 16. The padded surface of the cheek pad is towards the patient's cheek, thus facing down in this diagram. The four The hook and loop fastening system of VELCRO(™) arms are then folded at the dashed lines inwards on top of the attaching end 28 where they are coupled to the opposite The hook and loop fastening system of VELCRO(™) arm.

The advantage of the cheek pad in FIGS. 8A and 8B is that they are removable. However, it should also be apparent that the cheek pads could be constructed so as to be integral with the attaching ends.

It is to be understood that the above-described arrangements are only illustrative of the application of the principles of the present invention. Numerous modifications and alternative arrangements may be devised by those skilled in the art without departing from the spirit and scope of the present invention. The appended claims are intended to cover such modifications and arrangements.

1. An endotracheal tube holder for neo-natal use, said endotracheal tube holder comprising:
an arcuate face plate which is disposed adjacent to and in front of a patient's mouth, wherein the face plate includes a receptacle disposed so as to be relatively centered in front of the mouth, and wherein the face plate has a first attaching arm and a second attaching arm which are contiguous with a patient's cheeks as they extend from either side of the face plate towards a back of a patient's head;
an endotracheal tube holder member disposed in the front of the arcuate face plate, and which is configured for positioning an endotracheal tube away from a patient's palate;
a bite block disposed so as to be held by the endotracheal tube holder member, and which extends inwards into the patient's mouth, wherein the bite block is surrounded by a rubber sleeve so as to cushion gums and teeth as they are pressed against the bite block; and
a plurality of non-elastic attachment straps which are coupled to the first and the second attaching arms of the face plate, and which extend around the neo-natal patient's head so as to secure the face plate in front of the patient's mouth.

2. The endotracheal tube holder as defined in claim 1 wherein the endotracheal tube holder member further comprises:
a bottom member generally formed as half a cylinder cut lengthwise, which includes a first semicircular depression along a long axis for receiving the endotracheal tube; and
a top member generally formed as half a cylinder cut lengthwise, which includes a second semicircular depression for receiving the endotracheal tube, wherein the bottom member is coupled to the top member along a pivoting edge which is parallel to the lengthwise axis, such that the bottom member and the top member form a cylinder with a hole formed from the first and the second semicircular depressions through the center thereof.

3. The endotracheal tube holder as defined in claim 2 wherein the endotracheal tube holder member further comprises a bore hole through the top member so as to be parallel to the lengthwise axis, such that a second tube can be inserted through the bore hole, to thereby introduce fluids into the patient at the same time that the endotracheal tube is being utilized.

4. The endotracheal tube holder as defined in claim 2 wherein the endotracheal tube holder member further comprises a bore hole through the bottom member so as to be parallel to the lengthwise axis, such that a second tube can be inserted through the bore hole, to thereby introduce fluids into the patient at the same time that the endotracheal tube is being utilized.

5. The endotracheal tube holder as defined in claim 2 wherein the endotracheal tube holder member further comprises the bite block which is formed from the top member and the bottom member, and which extends inwards into the patient's mouth.

6. The endotracheal tube holder as defined in claim 2 wherein the endotracheal tube holder member further comprises:
the top member having a ratcheting hook; and
the bottom member having a plurality of complementary receiving indentations against which the ratcheting hook is able to grip, thereby enabling the top member and the bottom member to hold endotracheal tubes of varying diameters within the first and the second semicircular depressions in accordance with how far the ratcheting hook is pressed into the receiving indentations.

7. The endotracheal tube holder as defined in claim 1 wherein the endotracheal tube holder member further comprises:
a first cheek pad disposed between the first attaching arm and the patient's cheek; and
a second cheek pad disposed between the second attaching arm and the patient's cheek, to thereby cushion the patient's cheek from the first and the second attaching arms.

8. The endotracheal tube holder as defined in claim 1 wherein the plurality of non-elastic attachment straps are coupled to the first and the second attaching arms using using a hook and loop fastening system such as VELCRO™, to thereby enable easy and rapid adjustments thereof.

9. The endotracheal tube holder as defined in claim 8 wherein the plurality of non-elastic attachment straps further comprises:
a first strap which is coupled to the first and the second attaching arms, and which extends around a base of a neck of the patient;
a second strap which is coupled to the first attaching arm, extends upwards in a curve along the patient's cheek to a patient's forehead, and then back down the patient's other cheek where it is coupled to the second attaching arm;
a third and a fourth strap which are coupled to the first strap at the base of the neck, and which extend forwards over the head so as to be coupled to the second strap at approximately the patient's forehead; and
a fifth strap which is coupled to the second strap generally above the patient's eyes and in a generally horizontal position.

10. The endotracheal tube holder as defined in claim 9 wherein the plurality of non-elastic attachment straps further comprises:
a first strap which is coupled to the first and the second attaching arms, and which extends around a base of a neck of the patient;
a second strap which is coupled to the first attaching arm, extends upwards in a curve along the patient's cheek to a patient's forehead, and then back down the patient's other cheek where it is coupled to the second attaching arm;
a third and a fourth strap which are coupled to the first strap at the base of the neck, and which extend forwards over the head so as to be coupled to the second strap at approximately the patient's forehead; and
an eye covering which is disposed across the patient's eyes, and which has the second strap threaded through the eye covering on either side of the patient's eyes.

11. The endotracheal tube holder as defined in claim 9 wherein the plurality of non-elastic attachment straps further comprises:
a first strap which is coupled to the first and the second attaching arms, and which extends around a base of a neck of the patient;
a second strap which is coupled to the first attaching arm, extends upwards in a curve along the patient's cheek to a patient's forehead, and then back down the patient's other cheek where it is coupled to the second attaching arm;
a third and a fourth strap which are coupled to the first strap at the base of the neck, and which extend forwards over the head so as to be coupled to the second strap at approximately the patient's forehead; and
a fifth strap which is coupled to the third and fourth straps in a horizontal position at a back of the patient's head and approximately opposite the patient's eyes.

12. The endotracheal tube holder as defined in claim 1 wherein the face plate is constructed of a clear polycarbonate material to thereby assist in visual inspection of the patient.

13. A method for providing an endotracheal tube holder for neo-natal use to thereby avoid damaging tissues and bones of the neo-natal patient, said method comprising the steps of:
(1) providing the endotracheal tube holder including an arcuate face plate, a receptacle centered thereon, a first attaching arm and a second attaching arm, a tube holder member which is configured for holding an endotracheal tube and disposed within the receptacle, a plurality of attachment straps which are coupled to the first and the second attaching arms of the face plate, and which extend around the neo-natal patient's head so as to secure the face plate in front of the patient's mouth;
(2) centering the tube holder member in front of the patient's mouth;
(3) providing a bite block as part of the tube holder member which extends inwards into the patient's mouth, wherein the bite block is surrounded by a rubber sleeve which functions so as to cushion the gums and teeth as they are pressed against the bite block, and wherein an endotracheal tube passes through the tube holder member so as not to make contact with the patient's gums or teeth; and
(4) constructing the plurality of attachment straps using a non-elastic material to thereby avoid creating pressure on the neo-natal patient's cranial structure.

14. The method as defined in claim 13 wherein the method further comprises the step of:
(1) forming a bottom member as half a cylinder cut lengthwise, which includes a semicircular depression along a long axis for receiving the endotracheal tube; and
(2) forming a top member generally as half a cylinder cut lengthwise, which includes a semicircular depression for receiving the endotracheal tube, wherein the bottom member is coupled to the top member along a pivoting edge which is parallel to the lengthwise axis, such that the bottom member and the top member form a cylinder with a hole formed from the semicircular depressions through the center thereof; and
(3) boring a hole parallel to the lengthwise axis through the top member to thereby enable a second tube to be inserted into the patient's mouth at the same time as an endotracheal tube.

15. The method as defined in claim 14 wherein the method further comprises the step of providing a bite block which extends into the patient's mouth from the top member and the bottom member, to thereby prevent damage to a patient's teeth, gums and palate.

16. The method as defined in claim 14 wherein the method further comprises the steps of:
(1) providing a ratcheting hook on the top member; and
(2) providing a plurality of complementary receiving indentations against which the ratcheting hook is able to grip, thereby enabling the top member and the bottom member to hold endotracheal tubes of varying diameters within the semicircular depressions in accordance with how far the ratcheting hook is pressed into the receiving indentations.

17. The method as defined in claim 16 wherein the method further comprises the steps of:
(1) providing a first cheek pad disposed between the first attaching arm and the patient's cheek; and
(2) providing a second cheek pad disposed between the second attaching arm and the patient's cheek, to thereby cushion the patient's cheek from the first and the second attaching arms.

18. The method as defined in claim 10 wherein the method further comprises the step of providing The hook and loop fastening system of a hook and loop fastening system such as VELCRO(™) on the plurality of attachment straps to thereby enable quick and easy adjustments to the endotracheal tube holder.

19. The method as defined in claim 12 wherein the method further comprises the step of constructing the endotracheal tube holder of a clear polycarbonate material to thereby assist in visual inspection of the patient.

20. A method for protecting gums, teeth and palate of a neo-natal patient when using an endotracheal tube holder, said method comprising the steps of:
(1) providing the endotracheal tube holder including an arcuate face plate, a receptacle centered thereon, a first attaching arm and a second attaching arm, an endotracheal tube holder member which is configured for holding an endotracheal tube and disposed within the receptacle, a plurality of attachment straps which are coupled to the first and the second attaching arms of the face plate, and which extend around the neo-natal patient's head so as to secure the face plate in front of the patient's mouth; and
(2) disposing a cushioned bite block on the tube holder member, enabling the bite block to extend into the patient's mouth and provide a surface for the gums and teeth to rest without causing damage thereto, wherein the endotracheal tube passes through the cushioned bite block so as not to touch the gums and teeth, and wherein the cushioned bite block centers the endotracheal tube in the neo-natal patient's mouth to thereby prevent injury to a palate therein.
